SEQUENCE LISTING (15 sequences)

SEQ ID NO: 1 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - protein, 422 aa, Homo sapiens
MGSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 4 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 5 - protein, 408 aa, Artificial
  Hypercytokine H-IL-6, Fischer et al. 1997
PPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPARGGGGSGGGGSVEPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 6 - protein, 543 aa, Artificial
  sIL-6R/IL-6 with linker EFM, Chebath et al. 1997
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVEFMPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - protein, 553 aa, Artificial
  sIL-6R/IL-6 with linker EFGAGLVLGGQFM, Chebath et al. 1997
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVEFGAGLVLGGQFMPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 8 - protein, 540 aa, Artificial
  sIL-6R/IL-6 without linker, Chebath et al., 1997
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 9 - protein, 520 aa, Artificial
  Fusion of human sIL-6R/IL-6 with 13 aa linker
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPARGGGGSGGGGSVEPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 525 aa, Artificial
  Fusion of human sIL-6R/IL-6 with 18 aa linker
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPARGGGGSGGGGSGGGGSVEPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - protein, 546 aa, Artificial [sequence omitted]

SEQ ID NO: 12 - protein, 13 aa, Artificial
  Peptide linker
EFGAGLVLGGQFM

SEQ ID NO: 13 - protein, 13 aa, Artificial
  Peptide linker
RGGGGSGGGGSVE

SEQ ID NO: 14 - protein, 18 aa, Artificial
  Peptide linker
RGGGGSGGGGSGGGGSVE

SEQ ID NO: 15 - DNA, 3792 bp, Artificial
  Expression cassette comprising the CMV immediate early promoter directing expression of Hyper-IL-6 and Neomycin separated by an IRES
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggactggacaattcgagctcggtacccaattcgagctcgcccggggatgcggccgctcgacgcatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctcgaggaggtggaggttctggaggtggaggttctgtcgagccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacgtcgagggatcttccatacctaccagttctgcgcctgcaggtcgcggccgcatcctctagagtcgacccgaccgcggacgctcgactgcaggaattaattccgcccccccccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

(19) United States
(12) Patent Application Publication
(10) Pub. No.: US 2011/0002898 A1
(43) Pub. Date: Jan. 6, 2011

(54) VACCINE COMPOSITIONS

(75) Inventor: Andrzej MacKiewicz, Poznan (PL)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: AGIRX LIMITED, CHALVINGTON, EAST SUSSEX (GB)

(21) Appl. No.: 12/865,178

(22) PCT Filed: Jan. 30, 2009
(86) PCT No.: PCT/EP09/00626

§ 371 Date: Sep. 17, 2010

(30) Foreign Application Priority Data
Jan. 31, 2008 (EP) PCT/EP2008/000780

(51) Int. Cl. A61K 35/12; A61P 35/00

(52) U.S. Cl. 424/93.7

(57) ABSTRACT

The invention relates to tumour therapy. In particular, the present invention relates to vaccine compositions comprising allogenic cells modified with hypercytokines for the treatment of cancer in general and for the treatment of melanoma in particular.

### FIELD OF THE INVENTION

The invention relates to tumour therapy. In particular, the present invention relates to vaccine compositions comprising allogenic cells modified with hypercytokines for the treatment of cancer in general and for the treatment of melanoma in particular.

### BACKGROUND OF THE INVENTION AND STATE OF THE ART

### Cytokines and Cytokine Receptors

IL-11 together with IL-6, Leukaemia Inhibitor Factor (LIF), Oncostatin M (OSM), Ciliary Neutrophic Factor (CNTF), Cardiotrophin 1 (CT-I) belongs to the family of hemopoietic cytokines (named IL-6-type or gp130 cytokines), which share structural similarity and a common receptor subunit (gp130) (Bazan et al., 1990). Although, each of the IL-6-type cytokines requires a specific (unique) receptor complex, at least one molecule of gp130 is always engaged. Initially a ligand (IL-6, IL-11, CNTF) binds specifically to its a non-signaling receptor subunit and next recruits the signaling receptor chain. IL-6 and IL-11 use a gp 130 homodimer for transducing the signal, while LIF, CNTF, CT-I utilize a heterodimer gp130/LIFR. OSM either recruits gp130/OSMR or gp130/LIFR heterodimers (reviewed in Heinrich et al., 2003, Bravo et al., 2000).

The tertiary structure of IL-6-type cytokines has been intensely investigated during recent years. Crystal structures have been determined for LIF (Robinson et al., 1994), CNTF (McDonald et al., 1995), IL-6 (Somers et al., 1997) and OSM (Deller et al., 2000). These studies revealed that each ligand exhibits the long chain four-helix bundle topology, which comprises four tightly packed α-helices (named A, B, C and D) ranging from 15 to 22 amino acids in length. The helices are connected in an up-up-down-down arrangement by the polypeptide loops. The A-B and C-D loops are relatively long as they connect parallel helices, whereas the B-C loop is shorter as it connects a pair of antiparallel helices. Detailed structural analysis and mutagenesis studies of IL-6-type cytokines have identified three receptor binding sites (termed I, II, III), which seem to be conserved among the gp130 family (reviewed in Bravo et al., 2000). Site I, which enables ligand to bind to its non-signaling receptor, is formed by amino acids from the C-terminal part of the A-B loop and the C-terminal residues of the D helix. Site II seems to be a universal gp130 binding site for all members of IL-6-type cytokines and consists of exposed residues on helices A and C. Site III is composed of an N-terminal half of helix D, the N-terminal part of the A-B loop and amino acid residues of the end of the C-D loop. This site is always occupied by the second signaling receptor: gp130, LIFR or OSMR, depending upon the identity of the ligand.

The receptors involved in IL-6-type cytokine signaling belong to the type I membrane proteins. They possess an extracellular N-terminus and one transmembrane domain (with the exception of CNTFR, which is linked to the membrane by a lipid anchor (Davis et al., 1991). Because of a common structural motif in their extracellular region, they are classified as cytokine receptor class I family (Bazan et al., 1990). This family is characterized by the presence of at least one cytokine binding homology domain (CHD) consisting of two fibronectin-type-III-like domains (FNIII) termed D2 and D3. The CHD is composed of approximately 200 amino acids, with four positionally conserved cysteine residues at the N-terminal domain and a characteristic conserved Trp-Ser-X-Trp-Ser (WSXWS) motif at the C-terminal domain. Additionally each receptor subunit contains an Ig-like domain, which is located at the N-terminus of the membrane-proximal CHD. The IL-6-type receptors are divided into two groups: α and β subunits. Receptors a (for IL-6, IL-11 and CNTF) are not involved in signal transduction. Subunits β, the signal transducing receptor chains, contain a considerably larger cytoplasmic part than α subunits and have three membrane-proximal FNIII domains that may play some role such as in stabilization and/or in orientation of the transmembrane receptor dimers (reviewed in Bravo et al., 2000, Heinrich et al., 2003). Besides the membrane bound IL-6-type receptor subunits, their soluble forms were found in biological fluids (reviewed in Marz et al., 1999). They are formed either by limited proteolysis (shedding) of membrane-bound receptors or by translation from differently spliced mRNA.

### Fusion Proteins of Cytokines and Cytokine Receptors

In order to increase and modify potential bioactivity of some molecular agents, the idea of linkage of two soluble naturally existing components has been postulated. Such fusion proteins have already been described. The separately encoded subunits of IL-12 (p35 and p40) have been connected by a polypeptide linker (Lieschke et al., 1997). Hyper-IL-6 is another example of a new designer agent, which consists of D2 and D3 domain of IL-6 R α chain connected to IL-6 via polypeptide linker (Fischer et al., 1997 and WO 97/32891). In the case of IL-6, it was observed that the effective concentration of IL-6 and sIL-6 R, which is needed for the stimulation of cells which lack membrane IL-6 R is very high (Rose-John et al., 1990). Furthermore, the average half-life of the IL-6/sIL-6 R complex might be shorter than the time needed to assemble the IL-6/sIL-6 R/gp130 complex (Wells et al., 1996). The stability of IL-6/sIL-6 R complex was enhanced by linking both components in order to create a fusion protein (Hyper-IL-6) (WO 97/32891). Hyper-IL-6 can directly bind to its signal transducing receptor subunit and enhance IL-6 activity. Hyper-IL-6 is a fully active fusion protein, which mediates response at 100 to 1000-fold lower dose compared to the combination of soluble IL-6 and sIL-6 R molecules (Fischer et al., 1997). In analogy, another superagonist has been designed for IL-6-type family named IL-11/R-FP (Pflanz et al., 1999). IL-11/R-FP was created by covalently linking D2 and D3 domains of IL-11 R (position L/109-G/318) with IL-11 (position P/29-L/199) using a 21 amino acid linker and demonstrated 50-fold higher activity in vitro than the combination of IL-11 and sIL-11 R. However, this construct was composed of truncated segments of the human IL-11 R and IL-11 and, thus, lacks naturally existing parts of the respective receptor and cytokine. Moreover, the artificial linker used is no naturally occurring sequence, which contributes to the immunogenicity of IL-11/R-FP when used for treatment of human patients. Another sIL-11R IL-11 fusion protein comprising larger parts of the full length sIL-11 was described in WO 2005/113591 and which exhibited advantageous properties if compared to IL-11/R-FP.

WO 99/02552 A2 (Yeda Research and Development Co. Ltd. (Revel M. et al.) “Chimeric interleukin-6 soluble receptor/ligand protein, analogs thereof and uses thereof”, published 21 Jan. 1999) relates to chimeric proteins comprising a fusion protein product of sIL-6R and IL-6 and biologically active analogs of such proteins. In these chimeric proteins sIL-6R may be directly fused to IL-6 or via specific linker peptides. WO 99/02552 A2 further discusses a potential use of said chimeric proteins or analogs as inhibitors of cancer cells. It is also contemplated to use said chimeric proteins for the preparation of a medicament for treating mammalian cancers, for enhancement of bone marrow transplantation, for increasing hematopoiesis, or for treating liver or neurological disorders. The specific fusion proteins sIL-6R/IL-6 and sIL-6RδVal/IL-6 produced and examined in the Example section of WO 99/02552 have also been studied in an article by Chebath et al. (1997).

A review article by Kallen K. J. et al. (1997) discusses the potential therapeutic applications of interleukin-6 hyperagonists and antagonists. Said therapeutic applications comprise haematologic disorders, solid malignancies, cardiac ischaemia and transplantation, bone disease, glomerulonephritis and amyloidosis, acquired immunodeficiency syndrome, rheumatic disorders, autoimmunity, burns and major trauma, anaemia, expansion of immature haematopoietic stem cells in bone marrow transplantation and tumour therapy, inducing thrombopoiesis and liver regeneration.

Fusion proteins as those described above which comprise a cytokine and its physiological receptor are sometimes also called “hypercytokines” due to their high activity at lower doses as compared to the individual cytokine and/or a mixture of the cytokine with its soluble receptor.

### Tumour Vaccines

The concept of therapeutic cancer vaccines is based on the knowledge that adaptive immunity can be primed and activated to specifically recognize and kill tumour cells. For the last 25 years, several vaccine studies have demonstrated immunological and clinical responses in selected patients, e.g. in renal cell cancer (Kubler & Vieweg 2006). Following the discovery of tumour-associated antigens or dendritic cells (DCs) along with the progress made in molecular biology and biotechnology, which provided recombinant cytokines and gene delivery systems, several strategies of tumour vaccination were proposed: tumour cell-based vaccines consisting of tumour cells admixed with a particular adjuvant (e.g., bacillus Calmette-Guerin, Corynebacterium parvum or IFNs); genetically modified tumour vaccines based on tumour cells expressing genes encoding immunostimulatory factors; and DCs modified with tumour-derived RNA, loaded with peptides/tumour lysates or fused with tumour cells.

The inventors of the present application studied tumour vaccines designed for mass scale production. Such tumour vaccines consist of established allogenic tumour cells which are irradiated and injected into tumour-bearing patients. In studies with tumour cells genetically modified to express hypercytokines the inventors surprisingly found that a composition comprising two different genetically modified tumour cell lines has a synergistic effect at least on the IL-2 and INF-γ production of peripheral blood lymphocytes. This increased production of IL-2 and INF-γ causes a beneficial shift of the immune response towards a Th1 immune response which is connected with cytotoxic activity. The compositions of the present invention comprising a first and a second allogenic cell line genetically modified to express the same or different hypercytokines will therefore be better suited as medicaments for the treatment of tumours as those known from the prior art.

### SUMMARY OF THE INVENTION

According to a first aspect the present invention relates to a composition comprising (1) one or more first cells modified to express a first hyper-cytokine, and (2) one or more second cells modified to express a second hyper-cytokine, wherein the one or more second cells are different from the one or more first cells.

According to a second aspect the present invention relates to a composition according to the first aspect for use in medicine.

According to a third aspect the present invention relates to a pharmaceutical composition comprising a composition according to the first or the second aspect additionally comprising pharmaceutically acceptable diluents, carriers, excipients, fillers, binders, lubricants, glidants, disintegrants, adsorbents, and/or preservatives.

According to a fourth aspect the present invention relates to the composition according to the first or second aspect or the pharmaceutical composition according to the third aspect for the treatment or prevention of cancer.

In a fifth aspect the present invention relates to the use of a composition according to the first or second aspect for the preparation of a pharmaceutical composition for the treatment or prevention of cancer.

### BRIEF DESCRIPTION OF THE DRAWINGS

FIG. 1 shows the T-cell proliferative response in an allogenic mixed tumour-lymphocyte reaction (AMTLR).

Column 1: spontaneous T-cell proliferation, i.e. without tumour cells;
column 2: spontaneous T-cell proliferation (without tumour cells) in the presence of IL-6;
column 3: spontaneous T-cell proliferation (without tumour cells) in the presence of H6;
column 4: T-cell proliferation in response to allogenic tumour cells;
column 5: T-cell proliferation in response to allogenic tumour cells in the presence of IL-6;
column 6: T-cell proliferation in response to allogenic tumour cells in the presence of H6.

FIG. 2 shows the effect of anti-IL-2 antibody on the T-cell proliferation in an allogenic mixed tumour-lymphocyte reaction (AMTLR).

Column 1: T-cell proliferation in response to allogenic tumour cells;
column 2: T-cell proliferation in response to allogenic tumour cells in the presence of IL-6; column 3: T-cell proliferation in response to allogenic tumour cells in the presence of H6;
column 4: T-cell proliferation in response to allogenic tumour cells with IL-2 neutralization;
column 5: T-cell proliferation in response to allogenic tumour cells in the presence of IL-6 with IL-2 neutralization;
column 6: T-cell proliferation in response to allogenic tumour cells in the presence of H6 with IL-2 neutralization.

FIG. 3 shows the cytokine secretion by Mich1-H6 and Mich2-H6 cells expressed as MFI and pg/ml.

FIG. 4 shows the cytokine secretion by PBLC isolated from healthy individuals expressed as MFI and pg/ml.

FIG. 5 shows results from the stimulation of PBLC cytokine production by Mich1-H6 cells, by Mich2-H6 cells, and by a combination of Mich1-H6 and Mich2-H6 cells. The results are expressed as MFI and in pg/ml.

### DETAILED DESCRIPTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

The term “hyper-cytokine” refers to a fusion protein comprising, essentially consisting or consisting of (a) a soluble part of a cytokine receptor, and (b) a cytokine which can bind under physiological conditions to said soluble part of a cytokine receptor and an optional peptide linker positioned between the soluble cytokine receptor and the cytokine. In preferred embodiments, said cytokine is GM-CSF, IL-6, IL-11, IL-15, anti-TGF, EPO, interferon, LIF, OSM, CNTF, CT-1. If the cytokine is located N-terminally with respect to the cytokine receptor it is preferred that the cytokine still comprises its secretion signal, which will be cleaved during maturation of the protein, i.e. the mature hypercytokine protein will not comprise the secretion signal. If the cytokine is located C-terminally with respect to the cytokine receptor it is preferred that the cytokine does not comprise its secretion signal. The term “soluble cytokine receptor” refers to a soluble fragment of the cytokine receptor, e.g. which lacks most or all of the membrane-spanning part and the cytosolic part and comprises most or all of the extracellular part of the cytokine receptor, as for example sIL-6R and sIL-11R. The receptor fragment is soluble, if it is not or essentially not inserted into the membrane of a mammalian cell, preferably a human cell, expressing the receptor fragment. If the cytokine receptor is located N-terminally with respect to the cytokine it is preferred that the cytokine receptor still comprises its secretion signal, which will be cleaved during maturation of the protein, i.e. the mature hypercytokine protein will not comprise the secretion signal. If the cytokine receptor is located C-terminally with respect to the cytokine it is preferred that the cytokine receptor does not comprise its secretion signal. As indicated above the hyper-cytokine optionally may comprise a peptide linker positioned between the cytokine receptor and the cytokine. Preferably, said peptide linker has a low immunogenicity or is non-immunogenic. More preferably, said peptide linker is non-immunogenic to human beings. In preferred embodiments, the soluble cytokine receptor is located at the amino-terminal part of the hyper-cytokine and the cytokine is located at the carboxy-terminal part of the hyper-cytokine.

The term “hyper-cytokine activity” refers to the activity of the fusion protein. While particularly preferred hypercytokines have based on the same molar amount a 100- to 1000-fold higher activity in the same assays as the cytokine on which they are based or as a mixture of the cytokine and the cytokine receptor, i.e. the unfused parts forming the hypercytokine, not every hypercytokine will show such a dramatic improvement, which will depend among others on the length of the parts of cytokine and soluble cytokine receptor included and the length of the protein linker, if any, present. Numerous assays are known to assess the activity of the respective cytokine which forms the basis for a hypercytokine that can be employed in the present invention. If the respective hypercytokine has at least 10-fold the activity of the natural occurring cytokine on which it is based (at the same molar amount) or as a mixture of the unfused parts of the cytokine and the soluble part of the cytokine receptor it is considered within the meaning of this invention to exhibit hypercytokine activity. Preferably, it has at the same molar amount at least 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 5000-fold, 550-fold, 600-fold, 650-fold, 700-fold, 750-fold, 800-fold, 850-fold, 900-fold, 950-fold or 1000-fold the activity of the cytokine on which it is based or of a combination of the cytokine and the soluble cytokine receptor. Suitable assay systems include, e.g. for IL-6 hypercytokine the induction of proliferation of BAF-3/cells as described in Fischer M. et al. (1997).

The expression “at least 90% sequence identity” used throughout the specification preferably refers to a sequence identity of at least 90%, at least 91%, at least 92%, at least 93%, at least 94%, at least 95%, at least 96%, at least 97%, at least 98%, or at least 99% to the respective reference polypeptide. In case where the reference sequence is not specified in comparison to which the sequence identity percentage is to be calculated, the sequence identity is to be calculated with reference to the longer of the two sequences to be compared. If the reference sequence is indicated the sequence identity is determined on the basis of the full length of the sequence indicated by SEQ ID. For example, a peptide sequence consisting of 21 amino acids compared to the amino acids of full length IL-6 according to SEQ ID NO: 2 may exhibit a maximum sequence identity percentage of 9.9% (21:212) while a sequence with a length of 106 amino acids may exhibit a maximum sequence identity percentage of 50% (106:212).

The similarity of nucleotide and amino acid sequences, i.e. the percentage of sequence identity, can be determined via sequence alignments. Such alignments can be carried out with several art-known algorithms, preferably with hmmalign (HMMER package, http://hmmer.wustl.edu/) or with the CLUSTAL algorithm (Thompson J. D. et al., 1994) available e.g. on http://www.ebi.ac.uk/clustalw/ or on http://npsa-pbil.ibcp.fr/cgi-bininpsa_automat.pl?page=/NPSA/npsa_clustalw.html. Preferred parameters used are the default parameters as they are set on http://www.ebi.ac.uk/clustalw/index.html#. The grade of sequence identity (sequence matching) may be calculated using e.g. BLAST, BLAT or BlastZ (or BlastX). Preferably, sequence matching analysis may be supplemented by established homology mapping techniques like Shuffle-LAGAN (Brudno M., 2003) or Markov random fields. When percentages of sequence identity are calculated in the context of the present invention, these percentages are to be calculated in relation to the full length of the longer sequence, if not specifically indicated otherwise.

A “peptide linker” in the context of the present invention refers to an amino acid sequence of between 1 and 100 amino acids. In preferred embodiments, a peptide linker according to the present invention has a minimum length of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In further preferred embodiments, a peptide linker according to the present invention has a maximum length of 100, 95, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, or 15 amino acids or less. In especially preferred embodiments, the above-indicated preferred minimum and maximum lengths of the peptide linker according to the present invention may be combined, if such a combination makes mathematically sense. In further preferred embodiments, the peptide linker of the present invention is non-immunogenic; in particularly preferred embodiments, the peptide linker is non-immunogenic to humans.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a composition comprising, essentially consisting or consisting of:

(1) one or more first cells modified to express a first hyper-cytokine, and
(2) one or more second cells modified to express a second hyper-cytokine,
wherein the one or more second cells are different from the one or more first cells.

The second cells are different from the first cells, if the first and the second cells are derived from different cell lines and/or if the second cells carry a different genetic modification than the first cells, e.g. first and second cells have been modified to express different hypercytokines. In preferred embodiments the first and second cells, respectively, are derived from tissue of two different individuals, preferably from two different humans. It is preferred that the two tissues, preferably tumour tissues are of the same type. The term “tissue” as used herein refers to both solid tissue like, e.g. skin, liver, brain, kidney, lung, stomach, colon, bladder, or testes, as well as mobile cell populations like, e.g. lymphocytes, or stem cells. While it is possible that the cells are autologous or allogenic, it is particularly preferred that the first and/or the second cells are allogenic. The term “allogenic” characterizes the relation between the cells and a patient receiving the cells. Cells from a particular individual will be allogenic to any other patient, while they will be autologous to that particular individual. Allogenicity is a prerequisite for industrial large scale production of any cell based vaccine, since otherwise each cellular vaccine would have to be produced individually from cells isolated and cultured from the respective patient to be treated. Allogenic cells provide additional advantages, which include that allogenic cells tend to induce a stronger immune response in a patient than autologous cells.

The terms “one or more first cells” and “one or more second cells” as used in the present invention refer to an individual cell, to a clonal population of that cell and to an assortment of similar cells. Thus, in a preferred embodiment, wherein the cells are derived from primary tissue, preferably a primary tumour, the cells will not all be clonal, but will be composed of one, two, three or more clonal cell populations belonging to a particular cell and/or tumour type. In particular, tumour cells show a high genetic variability upon propagation and, thus, it is common that cells within one established cell line are not entirely identical genetically. These cells are an example of an assortment of similar cells. Another example are primary tumour cells originating from one tumour, which have undergone 1, 2, 3, 4, 5, 6, 7, 8, 9, 10 or more cycles of subcultivation in vitro, which will lead to selection of proliferating cell subtypes and, thus, to reduction of heterogeneity of the cell population, i.e. render the assortment of cells more similar. In an embodiment, wherein the first and/or second cells are derived from primary tissue, preferably the same type of primary tissue, in particular tumour tissue, the first and second cells are considered different, if they are derived from two different individuals, preferably from two different humans.

The term “modified to express” indicates that a gene encoding the respective hyper-cytokine has been stably introduced into the cell in a form which allows stable expression of the gene encoding the hypercytokine and, subsequently, production of the respective hypercytokine.

Preferably the gene encoding the hypercytokine is introduced into an expression vector for use in mammalian cells, which ordinarily include an origin of replication (as necessary, see below), a promoter located in front of the gene to be expressed, optionally an enhancer in trans, along with any necessary ribosome binding sites, RNA splice sites, polyadenylation site, and transcriptional terminator sequences. Such an expression vector may then be used to modify the cell to express the respective hypercytokine.

In a preferred embodiment the expression vector of the present invention comprises, essentially consists or consists of plasmids; phagemids; phages; cosmids; artificial chromosomes, in particular artificial mammalian chromosomes or artificial yeast chromosomes; knock-out or knock-in constructs; viruses, in particular adenovirus, vaccinia virus, attenuated vaccinia virus, canary pox virus, lentivirus (Chang and Gay, 2001), herpes virus, in particular Herpes simplex virus (HSV-1, Carlezon, et al., 2000), baculovirus, retrovirus, adeno-associated-virus (AAV, Carter and Samulski, 2000), rhinovirus, human immune deficiency virus (HIV), filovirus, and engineered versions of above mentioned viruses (see, for example, Kobinger et al., 2001); virosomes; “naked” DNA, liposomes; virus-like particles; and nucleic acid coated particles, in particular gold spheres. Particularly preferred are viral vectors like adenoviral vectors, lentiviral vectors, baculovirus vectors or retroviral vectors (Lindemann et al., 1997, and Springer et al., 1998). Examples of plasmids, which allow the generation of such recombinant viral vectors include pFastBac1 (Invitrogen Corp., Carlsbad Calif.), pDCCMV (Wiznerowicz et al., 1997) and pShuttle-CMV (Q-biogene, Carlsbad, Calif.). In cases where an adenovirus is used as an expression vector, the coding sequences may be ligated to an adenovirus transcription/translation control complex, e.g., the late promoter and tripartite leader sequence. The hypercytokine gene may be inserted in the genome of an adenovirus by in vitro or in vivo recombination. Insertion in a non-essential region of the viral genome (e.g., region E1, E3, or E4) will result in a recombinant virus that is viable and capable of expressing the respective hypercytokine in infected cells. It is preferred that the viral vector used is modified to be replication incompetent in order to prevent that first and/or second cells modified to express the hypercytokine produce viral particles.

To allow stable expression of a transgene the expression vector either has to be provided with an origin of replication, which allows replication independent from the genome of the cell or has to be integrated into the genome of the first and/or second cells. In the first case the expression vector is maintained episomally. Suitable origins of replication may be derived from SV40 or other viral (e.g., Polyoma, Adeno, CMV, VSV, BPV) source. In the latter case, if the expression vector is integrated into the genome, e.g. a chromosome, it is not required to provide an origin of replication.

To direct expression of the hypercytokine the gene encoding it is operationally linked to a promoter and/or enhancer that is recognized by the transcriptional machinery of the cell. Suitable promoters may be derived from the genome of mammalian cells (e.g., metallothionein promoter) or from mammalian viruses (e.g., the adenovirus late promoter; the vaccinia virus 7.5K promoter or the cytomegalovirus promoter). The early and late promoters of SV40 virus are particularly useful because both are obtained easily from the virus as a fragment which also contains the SV40 viral origin of replication. Smaller or larger SV40 fragments may also be used, provided there is included the approximately 250 by sequence extending from the HindIII site toward the BglII site located in the viral origin of replication. Further, it is also possible, and may be desirable, to utilize promoter or control sequences normally associated with the cytokine or cytokine receptor encoding polynucleotide on which the hypercytokine is based.

As used herein, “operatively linked” means incorporated into a genetic construct so that expression control sequences effectively control expression of a coding sequence of interest.

Specific initiation signals may also be required for efficient translation of hypercytokine coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators. In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

As indicated above rather than using expression vectors that contain viral origins of replication, cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and are then switched to a selective medium. The selectable marker in the recombinant plasmid confers resistance to the selection and allows cells to stably integrate the plasmid into their chromosomes and grow to form foci which in turn can be cloned and expanded into cell lines.

A number of selection systems may be used including, but not limited to, the herpes simplex virus thymidine kinase (tk), hypoxanthine-guanine phosphoribosyltransferase (hgprt) and adenine phosphoribosyltransferase (aprt) genes, in tk-, hgprt- or aprt-cells, respectively. Also antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin.

In a preferred embodiment the expression vector used to transform, transfect or infect the cell to be modified comprises the gene encoding the selectable marker as one transcript with the gene encoding the hypercytokine. To ascertain the individual expression of the selectable marker and the hypercytokine an internal ribosome entry site (IRES) is placed between the two coding sequences.

The cells to be included in the composition of the present invention are preferably propagated separately. Preferably they are propagated in vitro in one of two modes: as non-anchorage dependent cells growing in suspension throughout the bulk of the culture or as anchorage-dependent cells requiring attachment to a solid substrate for their propagation (i.e., a monolayer type of cell growth). The appropriate growth conditions are determined by the cell type and can be determined by the skilled person using routine experimentation.

In a preferred embodiment of the composition of the first aspect, the first and/or second hyper-cytokine is a fusion protein comprising, consisting essentially of or consisting of a soluble cytokine receptor and a cytokine. In preferred embodiments, the soluble cytokine receptor is independently selected from (a) the group consisting of sIL-6R, sIL-11R, sOSM-R, sCNTF-R, and sCT-1-R; or (b) a polypeptide exhibiting at least 90% sequence identity to a polypeptide according to (a); and the cytokine is independently selected from (c) the group consisting of IL-6, IL-11, OSM, CNTF, and CT-I; or (d) a polypeptide exhibiting at least 90% sequence identity to a polypeptide according to (c), and optionally a peptide linker between the soluble cytokine receptor and the cytokine, wherein the resulting fusion protein has hyper-cytokine activity. Preferably the arrangement is from the N-terminal end to the C-terminal end of the fusion protein as follows: soluble cytokine receptor-optional peptide linker-cytokine. To ascertain secretion of the expressed hypercytokine the hypercytokine comprises at least one natural or artificial secretion signal. Since all cytokines are secreted they naturally comprise such a secretion signal. Similar signalling peptides are also found in cytokine receptors. Preferably this secretion signal is located at the N-terminal end of the fusion protein. It will be cleaved during processing and/or secretion of the hypercytokine.

When referring to sIL-6R, sIL-11R, sOSM-R, sCNTF-R, and sCT-1-R the respective soluble parts of IL-6R, IL-11R, OSM-R, CNTF-R, and CT-1-R, preferably of human origin are meant, the sequence of which are indicated herein as SEQ ID NO: 1 for IL-6R and SEQ ID NO: 3 for IL-11R. The sequences of all other cytokine receptors can be accessed on NIH Genebank or EMBL databanks, e.g. for OSM-R (Genebank Acces.: NP_{—}003990) and CNTF-R (Genebank Acces.: NP_{—}001833). When referring to IL-6, IL-11, OSM, CNTF, and CT-I, preferably those of human origin are meant, the sequence of which are indicated herein as SEQ ID NO: 2 for IL-6 and SEQ ID NO: 4 for IL-11. The sequences of all other cytokines can be accessed on NIH or EMBL databanks, e.g. for OSM (Genebank Acces. NO: P13725) and CNTR (Genebank Acces.: NP_{—}000605).

In one embodiment of the composition of the first aspect, the hyper-cytokine is a fusion protein comprising, consisting essentially of or consisting of:
(a) an IL-6R part exhibiting at least 90% sequence identity to human soluble IL-6 receptor (sIL-6R), wherein said sequence identity is calculated over the entire length of the polypeptide sequence from P113 to A323 of SEQ ID NO: 1,
(b) an IL-6 part exhibiting at least 90% sequence identity to human interleukin-6 (IL-6), wherein said sequence identity is calculated over the entire length of the polypeptide sequence from P29 to M212 of SEQ ID NO: 2, and
(c) an optional peptide linker;

wherein the fusion protein has hyper-cytokine activity, preferably at least 10%, at least 20%, at least 30%, at least 40%, at least 50%, at least 60%, at least 70%, at least 80%, at least 90%, at least 100% or more of the activity of the Hyper-IL-6 fusion protein according to SEQ ID NO: 9, when tested in a relevant assay of IL-6 activity, e.g. the induction of proliferation of BAF-3/cells as described in Fischer M. et al. (1997).

In a further embodiment of the composition of the first aspect, the hyper-cytokine is a fusion protein comprising, consisting essentially of or consisting of:
(a) an IL-11R part exhibiting at least 90% sequence identity to human soluble IL-11 receptor (sIL-11R), wherein said sequence identity is calculated over the entire length of the polypeptide sequence from M1 to G365 of SEQ ID NO: 3,
(b) an IL-11 part exhibiting at least 90% sequence identity to human interleukin-11 (IL-11), wherein said sequence identity is calculated over the entire length of the polypeptide sequence from A19 to L199 of SEQ ID NO: 4, and
(c) an optional peptide linker;
   wherein the fusion protein has hyper-cytokine activity, preferably at least 10%, at least 20%, at least 30%, at least 40%, at least 50%, at least 60%, at least 70%, at least 80%, at least 90%, at least 100% or more of the activity of the sIL-11R-1L-11 fusion protein according to SEQ ID NO: 11, when tested in a relevant assay of IL-11 activity.

In a further preferred embodiment of the composition of the first aspect, the hyper-cytokine comprises, consists essentially of or consists of:
(a) a polypeptide having the amino acid sequence according to SEQ ID NO: 5 to 11; or
(b) a polypeptide exhibiting at least 90% sequence identity to a polypeptide according to (a) and having hyper-cytokine activity.

The polypeptide having the amino acid sequence according to SEQ ID NO: 5 consists of the part from P113 to A323 of IL-6R according to SEQ ID NO: 1, a glycine-rich linker sequence of 13 amino acids, and the part from P29 to M212 of IL-6 according to SEQ ID NO: 2 (Fischer et al., 1997). Further sIL-6R and IL-6 fusion proteins having the amino acids sequences according SEQ ID NO: 6, 7 and 8 have been described by Chebath et al. and consist of the region from M1 to V356 of IL-6R according to SEQ ID NO: 1, the region from P29 to M212 of IL-6 according to SEQ ID NO:2, and different linker sequences. The polypeptide according to SEQ ID NO: 6 comprises a 3 amino acid linker sequence (UM), the polypeptide according to SEQ ID NO: 7 comprises a 13 amino acid linker sequence (EFGAGLVLGGQFM; SEQ ID NO: 12), and the polypeptide according to SEQ ID NO: 8 contains no linker sequence. Additional, fusion proteins have been described in WO 97/32891 and comprise amino acids M1 to A323 of sIL-6R according to SEQ ID NO: 1 and amino acids and P29 to M212 of IL-6 according to SEQ ID NO:2 linked by either a 13 amino acid linker sequence (RGGGGSGGGGSVE, SEQ ID NO: 13) according to SEQ ID NO: 9 or a 18 amino acid linker sequence (RGGGGSGGGGSGGGGSVE; SEQ ID NO: 14) according to SEQ ID NO: 10. The fusion protein having the amino acid sequence according to SEQ ID NO: 11 comprises the region from M1 to Q365 of sIL-11R and the region from A 19 to L199 of IL-11. The sIL-6R IL-6 fusion according to SEQ ID NO: 9 is a particularly preferred embodiment and is referred herein as “Hyper-IL-6” or “H6”. A preferred expression cassette comprising Hyper-IL-6 and the Neo selectable marker both under the control of the CMV immediate early promoter is provided as SEQ ID NO: 15. This cassette may be comprised in a variety of vectors, preferably viral vectors like retroviral vectors.

In a preferred embodiment of the first aspect, the second cell, preferably the allogenic second cell, has a different human leukocyte antigen (HLA) type than the first cell, preferably the allogenic first cell. The HLA system is the name used for the human major histocompatibility complex (MHC). The group of genes encoding this complex resides on chromosome 6, and encodes cell-surface antigen-presenting proteins and many other genes. The major HLA antigens are essential elements in immune function.

Different classes have different functions
(a) class I antigens (A, B & C)—Present peptides from inside the cell (including viral peptides if present), and
(b) class II antigens (DR, DP, & DQ)—Present phagocytosed antigens from outside of the cell to T-lymphocytes
   Aside from the genes encoding the 6 major antigens, there are a large number of other genes, many involved in immune function located on the HLA complex. Diversity of HLA in human population is one aspect of disease defense, and, as a result, the chance of two unrelated individuals having identical HLA molecules on all loci is very low. The proteins encoded by HLAs are the proteins on the outer part of body cells that are (effectively) unique to that person.

The immune system uses the HLAs to differentiate self cells and non-self cells. Any cell displaying that individuals' HLA type belongs to that individual (and therefore is not an invader). Long before PCR based gene sequencing and gene identification were available, the HLA antigens were recognized as factors interfering with or, occasionally, permitting successful transplantion. Donor organs transplanted into recipients elicit antibodies against the donor's tissues and turning the donor's HLA receptors into antigens of the recipients immune system, hence the name ‘human leukocyte antigens’. The types of receptors could be classified based on the antibodies that they induced. These antibodies, particularly to donors who were homozygotes of a particular class II haplotype can be used to identify different receptor types and isoforms. There are two parallel systems of nomenclature that are used to classify HLA. The, first, and oldest system is based on serological (antibody based) recognition. In this system antigens are eventually assigned letters and numbers (e.g. HLA-B27 or, shortened, B27). A parallel system has been developed that allowed more refined definition of alleles, in this system a “HLA” is used in conjunction with a letter, an asterisk (*), and a four or more digit number (e.g. HLA-B*0801, A*68011, A*240201N N=Null) to designate a specific allele at a given HLA locus. HLA loci can be further classified into MHC class I and MHC class II (or rarely, D locus). This classification is based on sequence information from the respective HLA loci. Accordingly, the skilled person is well aware how to determine, whether two groups of cells have the same or a different HLA type. Preferably, the first and the second cell line have a different HLA type based on the antibody type classification system.

In preferred embodiments, the one or more first cells and/or the one and more second cells is a tumour cell. Preferably, the tumour cell is selected independently for each cell from the group consisting of a melanoma cell, a renal carcinoma cell, a prostate cancer cell, a colon cancer cell, a lung cancer cell, a pancreas cancer cell, a liver cancer cell, a brain cancer cell, a head and neck cancer cell, and a sarcoma cell. Preferably, the first and the second cells are selected from the same tumour cell type but either from different tumours within an individual or from two different individuals.

In one embodiment of the first aspect, the first cells, which are modified to express a hypercytokine are the human (Homo sapiens) melanoma derived cells Mich1, deposited on Apr. 24, 2007 under accession number DSM ACC2837 with the “Deutsche Sammlung von Mikroorganismen and Zellkulturen” (DSMZ), Inhoffenstr. 7 B, 38124 Braunschweig, Germany and/or the second cells, which are modified to express a hypercytokine are the human (Homo sapiens) melanoma derived cells Mich2, deposited on Apr. 24, 2007 under accession number DSM ACC2838 with the DSMZ. Mich1 and Mich2 originate from different patients.

In a preferred embodiment, the first cells are Mich1-H6, deposited on Apr. 24, 2007 under accession number DSM ACC2839 with the DSMZ. In a preferred embodiment, the second cells are Mich2-H6, deposited on Apr. 24, 2007 under accession number DSM ACC2840 with the DSMZ. Mich1-H6 and Mich2-H6 have respectively been derived from infection of Mich1 and Mich2 with a retrovirus comprising the expression cassette according to SEQ ID NO: 15 and expressing Hyper-IL-6 according to SEQ ID NO: 9 under the control of the CMV promoter.

The in vivo anti tumour effect exerted by compositions comprising first and second cells, in particular tumour cells, modified to express a hypercytokine can be further enhanced, if the first and/or second cells are engineered to comprise at least one further polynucleotide encoding an antigen, preferably a tumour antigen, a cytokine, in particular GM-CSF, IL-2, IL-6, IL-7, IL-11, IL-15, IL-21, anti-TGF, EPO, interferon, in particular INF-α, LIF, OSM, CNTF, CT-1 or a hypercytokine different from the first hypercytokine comprised in the respective cell. The engineering is preferentially achieved by using a vector, in particular one of the expression vectors indicated above with respect to hypercytokines and the subsequent or simultaneous introduction of this(ese) vector(s) into the first and/or second cells to be modified. The one or more additional polynucleotide can be comprised in a separate vector or can be comprised within the same vector as the hypercytokine encoding polynucleotide. It is preferred that the host cells simultaneously express both the hypercytokine and the at least one further protein encoded by the at least one further polynucleotide.

The term “tumour antigen” comprises all substances, which elicit an immune response against a tumour. Particular suitable substances are proteins or protein fragments which are enriched in a tumour cell in comparison to a healthy cell. These substances are preferably present within and/or are accessible on the outside of the tumour cell. If the tumour antigen is only present within a tumour cell, it will still be accessible for the immune system, since the antigen or fragments thereof will be presented by the MHC system at the surface of the cell. In a preferred aspect the tumour antigen is almost exclusively or exclusively present on and/or in the tumour cell and not in a healthy cell of the same cell type.

Suitable tumour antigens can be identified, for example, by analyzing the differential expression of proteins between tumour and healthy cells of the same cell type using a microarray-based approach (Russo et al., Oncogene. 2003, 22:6497-507), by PCR— or microarray-based screening for tumour specific mutated cellular genes (Heller, Annu. Rev. Biomed. Eng. 2002, 4:129-53) or by serological identification of antigens by recombinant expression cloning (SEREX; Tureci et al., Mol Med. Today. 1997, 3:342-349). The skilled artisan is aware of a large number of substances which are preferentially or exclusively present on and/or in a tumour cell, which include for example, oncogenes like, for example truncated epidermal growth factor, folate binding protein, melanoferrin, carcinoembryonic antigen, prostate-specific membrane antigen, HER2-neu.

Not all of the substances that are preferentially or exclusively present in and/or on a tumour cell will elicit a strong immune response, therefore, it is preferred that tumour antigens are selected to be expressed in the first and/or second cells of the composition of the invention, which elicit a strong immune response. Antigens eliciting a strong immune response will induce at least 1%, preferably at least 5%, more preferably at least 10% and most preferably at least 15% IFNγ-producing CD8+ T or CD4+ T cells isolated from mice previously immunized with the antigen, upon challenge with the antigen and/or will induce preferably at least 5%, and most preferably at least 15% of B-cells cells isolated from mice previously immunized with the antigen, upon challenge with the antigen to proliferate. Antigens fulfilling these criterions are candidates to be expressed in the cancer vaccine composition of the present invention.

In a particular preferred embodiment the tumour antigen is selected from the group consisting of T-cell-defined cancer-associated antigens belonging to unique gene products of mutated or recombined cellular genes, in particular cyclin-dependent kinases (e.g. CDC2, CDK2, CDK4), p15^{Ink4b}, p53, AFP, β-catenin, caspase 8, p53, p21^{Ras} mutations, Bcr-abl fusion product, MUM-1 MUM-2, MUM-3, ELF2M, HSP70-2M, HST-2, KIAA0205, RAGE, myosin/m, 707-AP, CDC27/m, ETV6/AML, TEL/Aml1, Dekcain, LDLR/FUT, Pml-RARα, TEL/AMLI; Cancer-testis (CT) antigens, in particular NY-ESO-1, members of the MAGE-family (MAGE-A1, MAGE-A2, MAGE-A3, MAGE-A4, MAGE-A6, MAGE-10, MAGE-12), BAGE, DAM-6, DAM-10, members of the GAGE-family (GAGE-1, GAGE-2, GAGE-3, GAGE-4, GAGE-5, GAGE-6, GAGE-7B, GAGE-8), NY-ESO-1, NA-88A, CAG-3, RCC-associated antigen G250; Tumour virus antigens, in particular human papilloma virus (HPV)-derived E6 or E7 oncoproteins, Epstein Barr virus EBNA2-6, LMP-1, LMP-2; overexpressed or tissue-specific differentiation antigens, in particular gp77, gp100, MART-1/Melan-A, p53, tyrosinase, tyrosinase-related protein (TRP-1 and TPR-2), PSA, PSM, MC¹R; widely expressed antigens, in particular ART4, CAMEL, CEA, CypB, HER2/neu, hTERT, hTRT, iCE, Muc1, Muc2, PRAME RU1, RU2, SART-1, SART-2, SART-3, and WT1; and fragments and derivatives thereof. Particular preferred tumour antigens are antigens derived from the tyrosinase-related protein.

When the composition of the present invention is administered to a patient it is administered to elicit an immune response both against the first and/or second cells and any tumour cells, which share epitopes and/or tumour antigens with the first and/or second cells. It is, thus, expected that the cells of the composition will only survive for a limited time within the recipient of the compositions of the present invention and are then cleared from the organism of the recipient by the immune system of the recipient. Nevertheless, it is preferred for safety reasons that the proliferation of the first, preferably allogenic and/or the second, preferably allogenic cells has been inhibited prior to the administration of these cells to a patient. The term “inhibition” comprises both the slowing down of the proliferation rate and the complete cessation of proliferation. The skilled person is aware of a larger number of chemical and physical methods, which affect the growth rate of cells, these include without limitation radiation, e.g. γ-irradiation or cross-linking, e.g. psoralen or aldehyde. The level of inhibition, however, should preferably be such, that transcription and translation of the transgenes introduced into the first and second cells is not completely shut down, i.e. the transgenes should be expressed at a level of at least 5%, preferably at least 10%, at least 20%, at least 30%, at least 40%, at least 50%, at least 60%, at least 70%, at least 80%, at least 90%, at least 100% or more of the first and/or second cells prior to inhibition. Preferably, the cells are capable to continue to go through 1 to 5, i.e. 1, 2, 3, 4, or 5, replication cycles after the chemical or physical method for inhibition of proliferation is administered.

The anti-tumour effect of the composition of the present invention can be further enhanced, if one or more additional cells, which may be autologous or allogenic, which are different from the first and/or the second cells are also included in the composition. Preferably these cells originate from a further individual, preferably having a HLA type different from the HLA types of the first and/or second cell types. Again it is preferred that these cells are tumour cells, preferably from the same tumour type as the first and/or second cells. For the reasons outlined above it is also preferred that the proliferation of the one or more additional, preferable allogenic cells has been inhibited, preferably as outlined above.

It is particularly preferred that the one or more cells of the one or more additional allogenic cells have been modified to express a cytokine, a cytokine receptor, a hypercytokine and/or a tumour antigene. Preferably, the cytokine is selected from the group consisting of GM-CSF, IL-2, IL-6, IL-7, IL-11, IL-15, IL-21, anti-TGF, EPO, interferon, in particular INF-α, LIF, OSM, CNTF, CT-1 and the cytokine receptors or soluble parts thereof are those receptors corresponding to the indicated cytokines. Preferably, the hypercytokine is selected from the group consisting of hyper-IL-6, e.g. according to SEQ ID NO: 5, 6, 7, 8, 9 or 10, IL-2, hyper-IL-11, e.g. according to SEQ ID NO: 11, hyper CNTF, and hyper-OSM.

As it is intended that the composition of the present invention elicits an immune response the composition may further comprise adjuvants, which are commonly used in vaccines to enhance the immunizing effect. Preferred adjuvants are selected from the group consisting of un-methylated DNA, in particular unmethylated DNA comprising CpG dinucleotides (CpG motif), in particular CpG ODN with phosphorothioate (PTO) backbone (CpG PTO ODN) or phosphodiester (PO) backbone (CpG PO ODN); gel-like precipitates of aluminum hydroxide (alum); bacterial products from the outer membrane of Gram-negative bacteria, in particular monophosphoryl lipid A (MPLA), lipopolysaccharides (LPS), muramyl dipeptides and derivatives thereof; synthetic lipopeptide derivatives, in particular Pam₃Cys; lipoarabinomannan; peptidoglycan; zymosan; heat shock proteins (HSP), in particular HSP 70; dsRNA and synthetic derivatives thereof, in particular Poly I:poly C; polycationic peptides, in particular poly-L-arginine; taxol; fibronectin; flagellin; imidazoquinoline; cytokines with adjuvant activity, in particular GM-CSF, interleukin-(IL-)₂, IL-6, IL-7, IL-18, type I and II, interferons, in particular interferon-gamma, TNF-alpha; oil in water emulsions, in particular MF59 consisting of squalene; Tween 80 and Span 85 (sorbitan-trioleate) and QS-21, a more highly purified derivative of Quil A, non-ionic block polymers, in particular Poloxamer 401, saponins and derivatives thereof, in particular the immunostimulatory fragments from saponins; polyphosphazene; N-(2-Deoxy-2-L-leucylamino-β-D-glucopyranosyl)-N-octadecyldodecanoylamide hydroacetate (BAY R1005), 25-dihydroxyvitamin D3 (calcitriol); DHEA; murametide [MDP(Gln)-OMe]; murapalmitine; polymers of lactic and/or glycolic acid; polymethyl methacrylate; sorbitan trioleate; squalane; stearyl tyrosine; squalene; theramide, synthetic oligopeptides, in particular MHCII-presented peptides. Particular preferred adjuvants, which can be comprised in the compositions of the present invention are selected from the group unmethylated DNA, in particular unmethylated DNA comprising CpG dinucleotides (CpG motif), in particular CpG ODN with phosphorothioate (PTO) backbone (CpG PTO ODN) or phosphodiester (PO) backbone (CpG PO ODN) and synthetic lipopeptide derivatives, in particular Pam₃Cys.

In a further aspect the present invention concerns a composition of the present invention for use in medicine.

In a further aspect the present invention concerns a pharmaceutical composition comprising a composition of the invention additionally comprising pharmaceutically acceptable diluents, carriers, excipients, fillers, binders, lubricants, glidants, disintegrants, adsorbents, and/or preservatives. Preferably the pharmaceutical composition is formulated for parenteral use, preferably in the form of a sterile aqueous solution which may contain other substances, for example, enough salts or glucose to make the solution isotonic with blood. The aqueous solutions should be suitably buffered (preferably to a pH of from 3 to 9), if necessary. A particularly preferred aqueous solution is phosphate buffered saline (PBS).

Preferably a unit dose of a composition of the present invention comprises between at least 1×10⁵ and 1×10⁹ cells of first cells, preferably at least 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×0⁷, 8×10⁷, 9×10⁷, and 1×10⁸. A particular preferred unit dose of a composition of the present invention comprises between 1×10⁷ to 1×10⁸ first cells, preferably 2.5×1×10⁷. Additionally, the unit dose of a composition of the present invention comprises between at least 1×10⁵ and 1×10⁹ cells of second cells, preferably at least 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, and 1×10⁸. A particular preferred unit dose of a composition of the present invention comprises between 1×10⁷ to 1×10⁸ second cells, preferably 2.5×1×10⁷. Preferably, the composition comprises about the same number of first cells and second cells for a total of 2×10⁵ to 2×10⁸ cells per unit dose. A particular preferred unit dose of a composition of the present, invention comprises between 2×10⁷ to 2×10⁸ first and second cells, preferably 5×10⁷. The total volume of the unit dose is preferably between 0.5 to 20 ml, preferably, 1 to 5 ml, e.g. 1, 2, 3, 4, or 5 ml.

In a further aspect the present invention relates to the composition of the present invention or the pharmaceutical composition of the present invention for the treatment or prevention of cancer. Preferred cancers treatable or preventable with a composition according to the present invention are selected from the group consisting of cancer of the gastrointestinal or colorectal tract, liver, pancreas, kidney, bladder, prostate, endometrium, head and neck cancer, ovary, testes, prostate, skin, eye, melanoma, dysplastic oral mucosa, invasive oral cancer, small cell and non-small cell lung cancer, hormone-dependent breast cancer, hormone independent breast cancer, transitional and squamous cell cancer, neurological malignancy, including neuroblastoma, glioma, astrocytoma, osteosarcoma, soft tissue sarcoma, hemangioma, endocrinological tumour, hematologic neoplasia including leukemia, lymphoma, and other myeloproliferative and lymphoproliferative diseases, carcinoma in situ, hyperplastic lesion, adenoma, and fibroma. Particular preferred is the treatment or prevention of melanoma, pancreatic and renal cancer.

In a further aspect the present invention relates to the use of a composition of the present invention for the preparation of a pharmaceutical composition for the treatment or prevention of cancer.

The compositions of the invention can be used in the treatment and/or prevention of a wide variety of different cancers, however, preferred cancers treatable or preventable according to the present invention are selected from the group consisting of cancer of the gastrointestinal or colorectal tract, liver, pancreas, kidney, bladder, prostate, endometrium, head and neck cancer, ovary, testes, prostate, skin, eye, melanoma, dysplastic oral mucosa, invasive oral cancer, small cell and non-small cell lung cancer, hormone-dependent breast cancer, hormone independent breast cancer, transitional and squamous cell cancer, neurological malignancy, including neuroblastoma, glioma, astrocytoma, osteosarcoma, soft tissue sarcoma, hemangioma, endocrinological tumour, hematologic neoplasia including leukemia, lymphoma, and other myeloproliferative and lymphoproliferative diseases, carcinoma in situ, hyperplastic lesion, adenoma, and fibroma. Particularly preferred is the treatment or prevention of melanoma, pancreatic and renal cancer. In particular in the context of the treatment and/or prevention of cancer it is envisionable that patients are immunized with a “cancer vaccine” prior to the development of any symptoms of a disease, i.e. receive a protective immunization, or after they have developed symptoms of the disease, i.e. receive a therapeutic vaccination.

The expression of at least one further cytokine, in particular GM-CSF, by the first and/or second cells expressing a hypercytokine, preferably Hyper-IL-6 can provide in the context of certain tumours, in particular melanoma and renal cancer an even stronger in vivo anti-tumour response than cells expressing only the hypercytokine. Therefore, in a preferred use the first and/or the second cells expressing hypercytokine are modified to express at least one further cytokine are used for the production of a medicament to prevent or treat a proliferative disease.

It is particularly preferred in this context when the first and the second cells are from the same type of tissue, preferably tumour tissue but have a partially or completely different HLA type than the first cell and/or the second cell.

### EXAMPLES

In the following, the invention is explained in more detail by non-limiting examples:

### Example 1

### Hyper-IL-6 (H6) Augments T-Cell Proliferative Response in Allogenic Mixed Tumour-Lymphocyte Reaction (AMTLR)

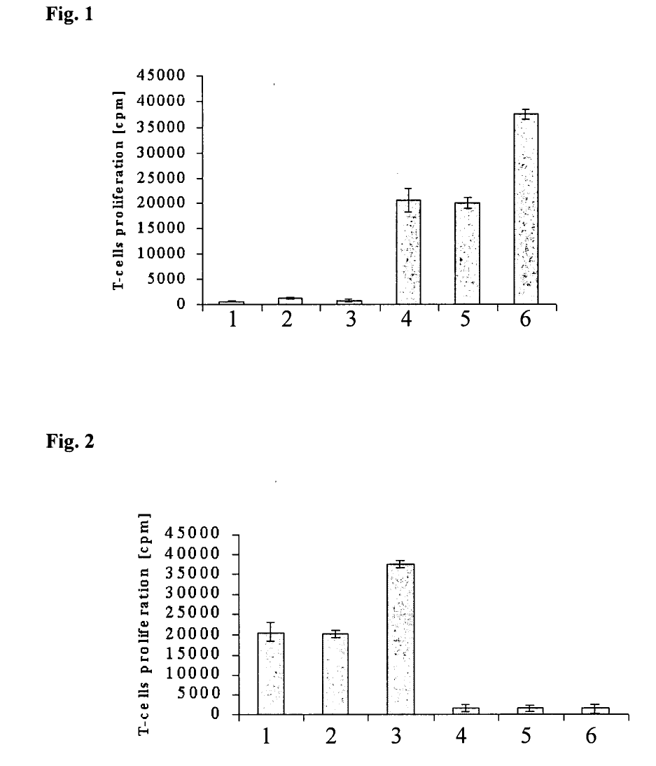

Irradiated tumour cells were mixed with unprimed, allogenic lymphocytes in the presence or absence of IL-6 (1 ng/ml) or purified H6 (1 ng/ml). After three days, T-cells were assayed for proliferation by ³H-thymidine incorporation, determined as counts per minute (cpm). The results of this experiment are shown in FIG. 1.

Columns 1 to 3 show the results of spontaneous T-cell proliferation, i.e. in the absence of tumour cells. Apparently, spontaneous T-cell proliferation does not occur to a significant extent (columns 1 to 3), irrespective whether IL-6 (column 2) or H6 (column 3) are added to the mixture.

Columns 4 to 6 show the results of T-cell proliferation in response to allogenic tumour cells. In the presence of allogenic tumour cells, the T-cells show very strong proliferation (column 4). The addition of IL-6 has no apparent effect on the proliferation of T-cells (column 5). The addition of H6 (column 6) leads to an almost two-fold increase in T-cell proliferation. Thus, H6 strongly enhances the T-cell proliferative response in an allogenic mixed tumour-lymphocyte reaction (AMTLR).

### Example 2

### T-Cell Proliferation in Allogenic Mixed Tumour-Lymphocyte Reaction is Dependent on IL-2

Irradiated tumour cells were mixed with unprimed, allogenic lymphocytes in the presence or absence of IL-6 (1 ng/ml), purified H6 (1 ng/ml) and anti-IL-2 antibody (1 μg/ml). After three days T-cells were assayed for proliferation by ³H-thymidine incorporation, determined as cpm. The results of this experiment are shown in FIG. 2.

Columns 1 to 3 of FIG. 2 show the results of T-cell proliferation in response to allogenic tumour cells in the absence of anti-IL-2 antibody. The results from this experiment are almost identical to the results presented in FIG. 1, columns 4 to 6. As shown in Example 1, T-cells show a very strong proliferation in the presence of allogenic tumour cells (column 1 of FIG. 2). The addition of IL-6 has no apparent effect on the proliferation of T-cells (column 2 of FIG. 2). The addition of H6 leads to an almost two-fold increase in T-cell proliferation (column 3 of FIG. 2).

Columns 4 to 6 of FIG. 2 show the results of T-cell proliferation in response to allogenic tumour cells in the presence of anti-IL-2 antibody, which neutralizes the effect of IL-2. The addition of the anti-IL-2 antibody greatly reduces T-cell proliferation (columns 4 to 6). The presence of IL-6 (column 5) or H6 (column 6) has no apparent effect on T-cell proliferation when anti-IL-2 antibody is present. Thus, the T-cell proliferation in the allogenic mixed tumour-lymphocyte reaction (AMTLR) is dependent on IL-2.

### Example 3

### Hyper-IL-6 Augments IL2 and IFN-γ Production by T-Cells in Allogenic Mixed Tumour-Lymphocytic Reaction

This example describes the evaluation of the immunostimulatory potential of Hyper-IL-6 (H6) in a mixed allogenic tumour/lymphocyte reaction. Results obtained indicate that Hyper-IL-6 increases immunostimulatory potential of allogenic melanoma cells. Moreover, Hyper-IL-6 is not only more potent than IL-6 but also displays qualitatively different biological activity. In contrast to native IL-6 which is a known Th2 inducer, Hyper-IL-6 appears to reduce IL-10 expression while increasing IFN-γ and IL-2 production by peripheral blood lymphocytes (PBLC) which is characteristic of a Th1 response.

Test Articles: A375 melanoma cells and their derivative A375-H6 cells; PBLC isolated from a healthy volunteer.

Media, components and equipment: FBS (GIBCO/Invitrogen), PBS (GIBCO/Invitrogen), DMEM (GIBCO/Invitrogen), Trypsin EDTA (GIBCO/Invitrogen), Tissue culture flask 25 cm² (Sarstedt), 24 well plate (Nunc), Lymphocyte separation medium (ICN), BD Cytometric Bead Array (CBA) Human Th1/Th2 Cytokine Kit-II (Becton Dickinson), IL-6 (Pharmingen), Flow cytometer (Becton Dickinson), FACSAria™ (Becton Dickinson).

Methods:

PBLC drawn from a healthy volunteer were separated from whole blood by centrifugation over lymphocyte separation medium. Cells were washed twice in PBS and counted by standard procedures in a haemocytometer. Lymphocytes were re-suspended at 2×10⁶ cells per ml in DMEM culture medium supplemented with 2% FBS. Tumour cells were trypsinized, washed twice in PBS and re-suspended at 2×10⁶ cells per ml in DMEM medium supplemented with 2% FBS. 0.5 ml of lymphocyte suspension was mixed with 0.5 ml of tumour cells and seeded on a 24 well plate to give 1 ml of mixed cell culture.

Experimental settings:

0.5 ml of lymphocyte suspension+0.5 ml of culture medium (control)
0.5 ml of lymphocyte suspension+0.5 ml of culture medium+10 ng of IL-6
0.5 ml of lymphocyte suspension+0.5 ml of A375 tumour cells suspension
0.5 ml of lymphocyte suspension+0.5 ml of A375 cells suspension+10 ng of IL-6
0.5 ml of lymphocyte suspension+0.5 ml of A375-H6 cells suspension
0.5 ml of A375 tumour cells suspension+0.5 ml of culture medium
0.5 ml of A375-H6 tumour cells suspension+0.5 ml of culture medium

The mixed cells were cultured for three days in a humidified cell incubator at 37° C., 5% CO₂/95% air. After 3 days cell-free supernatant was collected and analyzed for cytokine content. Collected supernatants were properly marked and immediately frozen at −20° C. until analysis. Cytokine content was determined by CBA within one month according to the instruction provided in the CBA manual.

The results of this example are summarized in Table 1 below. They show that the A375 tumour cells stimulate allogenic T-cells to produce IL-2, IL-6, IL-10 and IFN-γ. The presence of hyper-IL6 but not IL-6 significantly augmented IL-2 and IFN-γ production in T-cells and at the same time reduced IL-10 secretion.

TABLE 1
Cytokine production by allogenic tumour reactive T-cells

| | cytokines pg/ml | | | | | |
|---|---|---|---|---|---|---|
| | IL-2 | IL-4 | IL-6 | IL-10 | TNF-α | IFN-γ |
| A375 | 0 | 0 | 48 | 0 | 0 | 0 |
| A375-H6 | 0 | 0 | >5000 | 46 | 0 | 0 |
| PBLC | 3 | 0 | 583 | 11 | 0 | 0 |
| PBLC + | 2 | 0 | 283 | 7 | 0 | 0 |
| IL-6 | | | | | | |
| A375 + | 92 | 0 | >5000 | 113 | 0 | 19 |
| PBLC | | | | | | |
| A375 + | 96 | 0 | >5000 | 140 | 0 | 19 |
| IL-6 + | | | | | | |
| PBLC | | | | | | |
| A375- | 348 | 0 | >5000 | 78 | 0 | 106 |
| H6 + | | | | | | |
| PBLC | | | | | | |

From the above results it is apparent that hyper-IL-6 is not only more potent then IL-6 but also displays a qualitatively different biological activity. In contrast to native IL-6 which is a known Th2 inducer, hyper-IL-6 appears to reduce IL-10 expression while increasing IFN-γ and IL-2 production characteristic for Th1 response. This type of T-helper response (i.e. Th1) is of primary importance during cytotoxic T-cell induction and development and is therefore a desirable response in anti-tumour vaccines.

Without wishing to be bound by a single explanation, the inventors assume that the apparent lack of IL-6 activity added in excess into culture media can be explained as follows: From the above experiments it appears that allogenic tumour cells on their own induce very potent IL-6 production in reacting T-cells. This production reaches very high levels and most likely saturates all IL-6 specific receptors. As a result, addition of IL-6 into the culture system does not have any influence on T-cell behavior. On the other hand hyper-IL6 does not need any free IL-6 receptor, instead it binds to a common gp130 receptor subunit. Moreover, since hyper-IL6 does not need a specific IL-6 receptor it may operate on different cell populations compared to IL-6. The observed effects may be due to extra stimuli of IL-6 responding T-cells or independent T-cell stimulation which are negative for IL-6 receptor.

From the results presented in this Example it is apparent that under in vitro conditions hyper-IL-6 significantly increases stimulatory potential of human allogenic melanoma cells by shifting the immune response towards a Th1 type i.e. a cellular response.

### Example 4

### Synthesis of Cytokines, Growth Factors and Vascular Factors by Melanoma Cells and H6-Modified Melanoma Cells

The aim of this example was to assess the synthesis of selected cytokines, growth and vascular factors by melanoma cells which can be used as components in a vaccine. A further aim was to evaluate the effect of the H6 gene modification on the synthesis of above factors by said melanoma cells. Specific aims included (i) analysis of secretion of soluble factors such as IL-2, IL-4, IL-6, IL-8, IL-10, IL-12, INFγ, GM-CSF, RANTES and VEGF by melanoma cells Mich1 and Mich2; (ii) analysis of secretion of above factors by Mich1-H6 and Mich2-H6 cells (H6-modified melanoma cells); (iii) comparison of secretion pattern of H6-modified and parental Mich1 and Mich2 cells.

Test Articles Mich1 cells, Mich2 cells, Mich1-H6 cells and Mich2-H6 cells were thawed and cultured for two days and passaged for the next 3 days. Then cells were trypsinized and frozen. These cells (passage 1-P1) were used in the experiment.

TABLE 2
Deposit numbers of cell lines used

| | International | | Accession number given |
|---|---|---|---|
| Name of | Depository | Date of | by the International |
| cell line | Authority | Deposit | Depository Authority |
| Mich1 | DSMZ¹ | 24 April 2007 | DSM ACC2837 |
| Mich2 | DSMZ | 24 April 2007 | DSM ACC2838 |
| Mich1-H6 | DSMZ | 24 April 2007 | DSM ACC2839 |
| Mich2-H6 | DSMZ | 24 April 2007 | DSM ACC2840 |

¹ DSMZ - Deutsche Sammlung von Mikroorganismen and Zellkulturen GmbH, Inhoffenstr. 7 B, D-38124 Braunschweig, Germany

Methodology: The cell lines studied were thawed and seeded in culture flasks. Cells were cultured until confluency and then maintained in serum (FBS) free medium (DMEM) for 48 hr in 5% CO₂ humidified atmosphere. Then the media were collected and analyzed for the above-listed factors using multiplex particle-based immunoassay with FC readout.

### Secretion of Analyzed Factors by the Four Cell Lines Studied

Results of the amounts of the cytokines and growth factors secreted into the culture media by Mich1, Mich2 and Mich1-H6, Mich2-H6 cells are summarized in Table 3.

TABLE 3
Secretion of different cytokines and growth factors
by melanoma cells and H6-modifed melanoma cells

| Cytokine | Cells | | | |
|---|---|---|---|---|
| ng/ml | Mich1 | Mich1-H6 | Mich2 | Mich2-H6 |
| IL-2 | 0 | 0.3 | 0 | 0.2 |
| IL-4 | 0 | 0 | 0 | 0 |
| IL-6 | 0.2 | 44.8 | 0.8 | 29.7 |
| IL-8 | 11.1 | 89.3 | 13.5 | 64.5 |
| IL-10 | 0 | 0.02 | 0 | 0.01 |
| IL-12 | 0.03 | 0.04 | 0.05 | 0.03 |
| GM-CSF | 0.3 | 0.02 | 2.5 | 40 |
| RANTES | 0.05 | 0.6 | 0.04 | 0.6 |
| VEGF | 6.4 | 20.1 | 0.4 | 0.4 |
| IFN-γ | 0.02 | 0.07 | 0.06 | 0 |

Mich1 cells secreted low levels of IL-12, RANTES, and INF-γ; and moderate levels of IL-6 and GM-CSF; and relatively high levels of IL-8 and VEGF. IL-2, IL-4 and IL-10 were either not secreted or secreted at extremely low levels which were below the detection limit of the assay.

Mich2 cells secreted low levels of IL-12, RANTES and INF-γ; moderate levels of IL-6 and VEGF; and high levels of IL-8 and GM-CSF. IL-2, IL-4 and IL-10 were not detectable in the assay.

Mich1-H6 cells secreted low levels of IL-10, IL-12, GM-CSF and INF-γ; moderate levels of IL-2 and RANTES; high levels of IL-6 and VEGF; and very high levels of IL-8. IL-4 was not detectable in the assay.

Mich2-H6 cells secreted low levels of IL-10 and IL-12; moderate levels of IL-2, RANTES and VEGF; high levels of IL-6 and very high levels of IL-8 and GM-CSF. IL-4 and INF-γ were not detectable in the assay.

### Comparison of the Secretion Pattern of Studied Factors by Mich1 and Mich2 Cells

A comparison of the secretion pattern of the studied factors by parental cell lines Mich1 and Mich2 demonstrated significant qualitative similarities (see Table 3). Cells of both lines secreted IL-6, IL-8, IL-12, GM-CSF, RANTES, VEGF and INF-γ, but did not secrete IL-2, IL-4 and IL-10. However, some quantitative differences in GM-CSF and VEGF secretion between both lines were observed.

### Comparison of the Secretion Pattern of Studied Factors by Mich1-H6 and Mich2-H6 Cells

A comparison of the secretion pattern of studied factors by the modified cell lines Mich1-H6 and Mich2-H6 in general revealed qualitatively similar secretion patterns (see Table 3). Cells of both lines secreted IL-2, IL-6, IL-8, IL-10, IL-12, GM-CSF, RANTES and VEGF. Mich1-H6 cells secreted INF-γ, while Mich2-H6 did not. None secreted IL-4. Significant quantitative differences were seen for GM-CSF and VEGF secretion between both lines (3 and 2 orders of magnitude, respectively).

### Effect of the H6 Modification on the Secretion Pattern of Studied Factors by Melanoma Cell Lines

Mich1 cells: Significant qualitative (IL-2, IL-10) and quantitative differences between Mich1 and Mich1-H6 cells are observed. Except for GM-CSF which was decreased, expression of all other factors studied was significantly increased in H6 modified cells.

Mich2 cells: Significant qualitative (IL-2, IL-10, INF-γ) and quantitative differences between Mich2 and Mich2-H6 cells are observed. Except for IL-12 and VEGF which were at the same level, expression of all factors studied was significantly increased. In contrast, Mich2-H6 cells did not express INF-γ.

### Summary of Results from Example 4

Mich1 and Mich2 cells secreted 7 out of 10 factors studied. H6 modification resulted in the induction of 2 additional proteins (IL-2 and IL-10) and increased production of most of the other factors studied. Some factors such as IL-8, VEGF or GM-CSF were secreted by modified cells in the tenths of nanograms. IL-8 is a very strong chemoattractant increasing recruitment of immune cells into the vaccine injection site. GM-CSF is a major stimulator of dendritic cell maturation, hence inducing antigen presentation. VEGF is a signaling protein involved in vasculogenesis and angiogenesis. It is also capable of stimulating monocyte/macrophage migration. IL-2, IL-12 and INF-γ display immunomodulatory functions on T cells. Modified cells also secreted IL-10 which is considered to be an immunoinhibitory factor. However, experimental studies demonstrated that murine melanoma cells modified with IL-10 cDNA elicited specific anti-melanoma immune responses indicating that in such setting IL-10 provides a stimulatory signal for T lymphocytes. Moreover, modified cells secreted significant quantities of IL-6. However, additional identification studies are necessary since anti-IL-6 antibodies may react with H6 protein. Accordingly, high IL-6 levels detected by the employed method in the culture medium may reflect secretion of the transgenic H6 protein but not necessarily the native IL-6 protein by vaccine cells.

### Example 5

### Cytokine Production of a Mixture of Two H6-Modified Melanoma Cell Lines in an AMTLR

The aim of this example included the analysis of the effect of a mixture of two melanoma cell lines (Mich1-H6 and Mich2-H6) on cytokine production by PBLC isolated from various healthy individuals with different HLA haplotypes. Moreover, the effect of a mixture of two cell lines was compared with each line used alone. Cytokine production was assessed by measurement of cytokine content in culture medium by CBA (Cytometry Bead Assay).

Cells: Mich1-H6 cells and Mich2-H6 cells were obtained from the same source as described in Example 4 and were irradiated; PBLC were isolated from 4 healthy volunteers (3 males and 1 female).

Media, Components and Equipment: DMEM (GIBCO/Invitrogen); FBS (GIBCO/Invitrogen); PBS (GIBCO/Invitrogen); Lymphocytes Separation Medium (ICN); ³H-thymidine (Amersham Biosciences); Trypsin EDTA (GIBCO/Invitrogen); 96 well plate (Sarstedt); 75 ml tissue culture flasks (Corning); BD Cytometric Bead Array (CBA) Human Th1/Th2 Cytokine Kit-II (BD Biosciences); FACS Aria™ (BD Biosciences); Scintillation Counter; Phase-contrast microscope (Olympus); CO₂ Incubator (Sanyo); Centrifuge (Sorvall).

Cell preparation: Cells were cultured per standard procedures in the lab under GMP-Like conditions by a qualified research worker.

Mich1-H6 and Mich2-H6 cells were plated separately into culture flasks in DMEM culture medium supplemented with 10% FBS at a seeding density of approximately 1.67×10⁴ cells per cm². The cells were grown in culture until confluency (3-5 days) and were then trypsinized, washed twice with PBS, re-suspended at 2×10⁶ cells per ml in DMEM medium supplemented with 2% FBS for cytokine production record, then irradiated at 80Gy (60Co).

PBLCs were separated from the whole blood collected from 4 healthy individuals (coded: A, D, M and N) by centrifugation over lymphocyte separation medium. Cells were washed twice with PBS and counted by standard procedures in a haemocytometer. PBLCs were then re-suspended at 5×10⁵ cells per ml in DMEM culture medium supplemented with 2% FBS for cytokine production record.

### Set-Up of Cytokine Production Analysis

The test was performed on one 96 well plate for the IL-2, IL-4, IL-6, IL-10, TNF-α and IFN-γ cytokine production panel.

The cell suspension was transferred into the 96 well plate in a volume of 100 μl per each cell line in one row for each of four individuals. Into each row the cells were transferred in concentration of 2×10⁵ cells per well. Each cell line was reduplicated in four variant columns (four samples for each cell line). There were no melanoma cells added into the last but one 16 wells (4 per each of four individuals), i.e. in Row 5, with these wells acting as a negative control (Control −) for spontaneous PBLC proliferation (the control wells contained DMEM+2% FBS in quantity of 100 μl and PBLC in concentration of 5×10⁴ cells per well). There were no PBLCs added into the last 12 wells, i.e. in Row 6, with these wells acting as a positive control (Control +) for spontaneous melanoma cells proliferation (the positive control will contain DMEM+2% FBS in quantity of 100 μl and melanoma cells in concentration of 2×10⁵ cells per well for Mich1-H6, Mich2-H6 and mixture of both cell lines, respectively).

Then PBLCs were added to each well including the negative control wells (but except positive control wells) in a volume of 100 μl and at concentration of 0.5×10⁵ cells per well. The total volume in each well was 200 μl (i.e. 100 μl of PBLC plus 100 μl of melanoma cells solutions).

TABLE 4
Arrangement of samples and controls

| Cell | | | Michl-H6 + | |
|---|---|---|---|---|
| Component | Michl-H6 | Mich2-H6 | Mich2-H6 | Control |
| Row 1 | 2 × 10⁵ | 2 × 10⁵ | 1 × 10⁵ each | — |
| Row 2 | 2 × 10⁵ | 2 × 10⁵ | 1 × 10⁵ each | — |
| Row 3 | 2 × 10⁵ | 2 × 10⁵ | 1 × 10⁵ each | — |
| Row 4 | 2 × 10⁵ | 2 × 10⁵ | 1 × 10⁵ each | — |
| Control − | 5 × 10⁴ | 5 × 10⁴ | 5 × 10⁴ | 5 × 10⁴ |
| Control + | 2 × 10⁵ | 2 × 10⁵ | 1 × 10⁵ each | — |

The mixed cells were co-cultured for 3 days for cytokine production analysis, as it is optimal period for incubation, in a humidified incubator at 37° C. in 5% CO₂/95% air atmosphere.

After the predefined period of three days cell free supernatants from the plate were collected, properly marked and immediately frozen at −20° C. until further analysis. Cytokines accumulated in the medium were measured using BDTM Biosciences Cytometric Bead Array (CBA) Human Th1/Th2 Cytokine Kit-II Assay within one month according to the instruction provided in the CBA manual.

### Cytokine Secretion

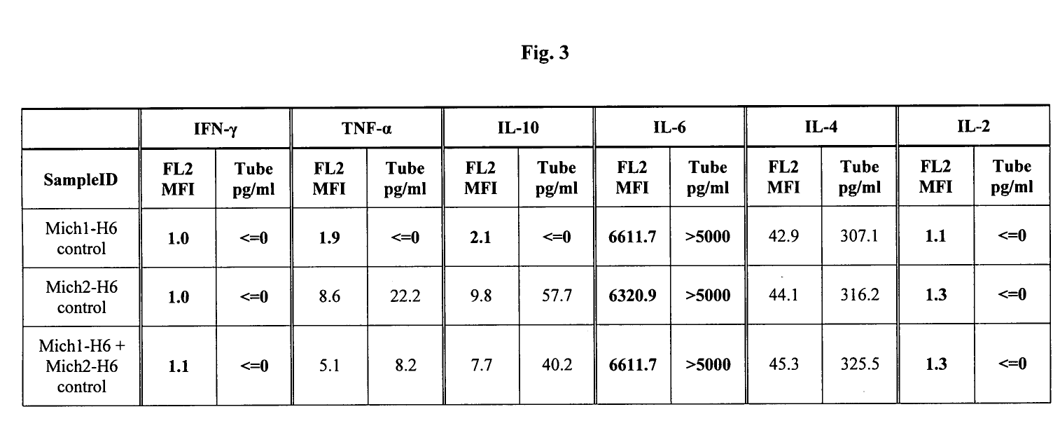

As shown in FIG. 3, Mich1-H6 cells did not produce IFN-γ, TNF-α, IL-10 and IL-2. Mich2-H6 cells did not produce IFN-γ and IL-2. As expected, these cytokines were also not seen in culture media of mixtures of both cell lines. However, low quantities of TNF-α and IL-10 were secreted by Mich2-H6 cells. Both cell lines produced IL-6 and IL-4 in very high and moderate quantities, respectively. These two cytokines were produced at comparable levels by each cell line.

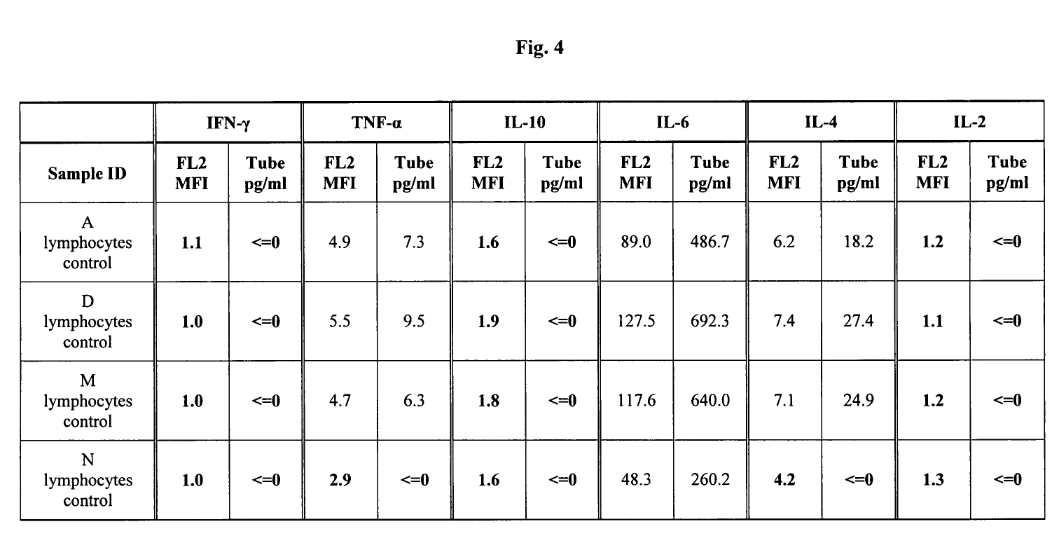

In FIG. 4 the results of cytokine production by PBLCs are shown. The PBLC did not produce IFN-γ, IL-10 or IL-2. The remaining cytokines examined, i.e. TNF-α, IL-6 and IL-4, were produced by PBLC at comparable levels.

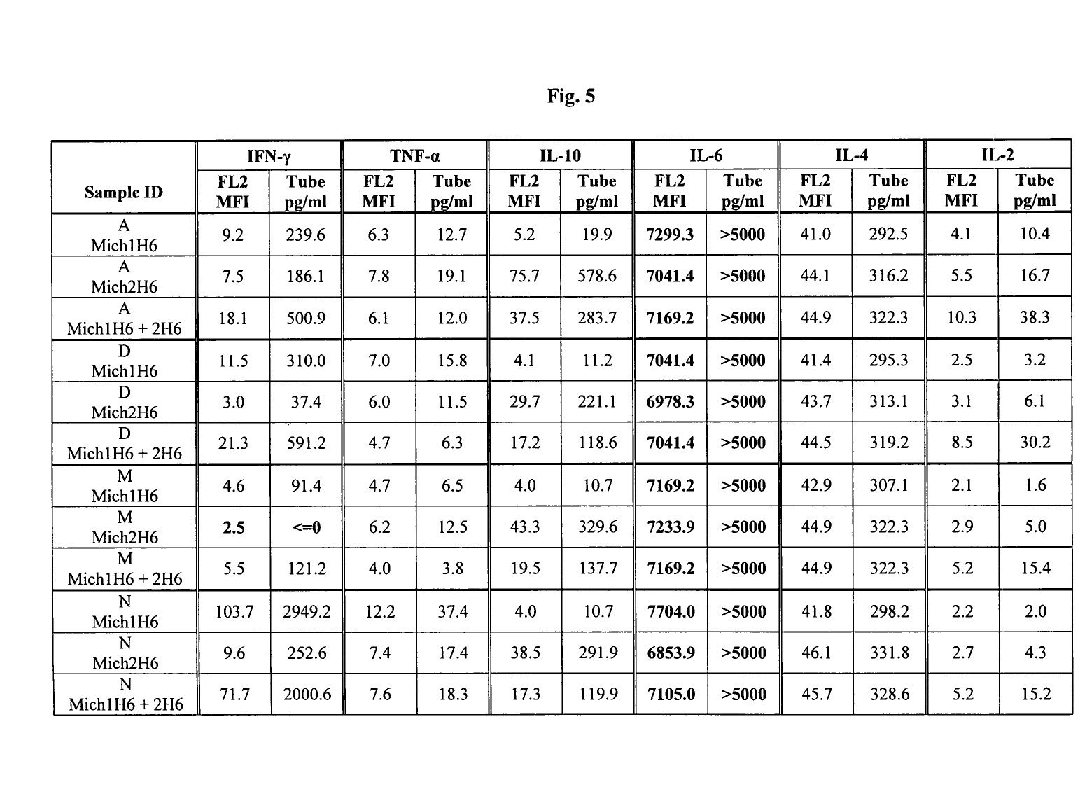

FIG. 5 shows results of the effect of Mich1-H6 and Mich2-H6 cells alone and in combination on PBLC cytokine secretion. Incubation of PBLC with melanoma cell lines led to the modulation of cytokine production by PBLC for most of the cytokines studied. Only IL-4 and likely IL-6 secretion were not affected. In PBLCs from all four donors, a synergistic effect of a mixture of both cell lines as compared to each cell line used separately was observed with respect to IL-2 production. A similar synergistic effect was seen in 3 out of 4 donors on IFN-γ production. An inhibitory effect of a mixture of cell lines over cells used separately was observed with regard to TNF-α production in PBLCs from 3 out of 4 donors. The production of IL-10 caused by the mixture of Mich1-H6 and Mich2-H6 was in between the values observed when PBLCs were stimulated by Mich1-H6 or Mich2-H6 separately.

### Summary of Results from Example 5

The results obtained demonstrate that the mixture of two allogenic melanoma cell lines displays different biological effects on cytokine secretion by PBLC isolated from healthy donors as compared to each cell line used separately. These effects proved to be synergistic on the increased production of IL-2 and INF-γ and inhibitory on TNF-α secretion. Increased IL-2 and INF-γ production indicate the beneficial shift towards a Th1 immune response. Decreased TNF-a secretion requires further studies since the cytokine quantities detected were near to the lower detection limit of the assay.

In conclusion, the combination of Mich1-H6 and Mich2-H6 cells increases in vitro immunogeneicity of the vaccine as compared to each cell line used alone by shifting the immune response towards a Th1 type as demonstrated by the synergistic effect on IL-2 and INF-γproduction by PBLC. Thus, the genetic modification of tumour cells with a designer cytokine, e.g. a hypercytokine such as H6, and the combination of multiple tumour cell lines increases the therapeutic potential of an allogenic vaccine.

### REFERENCES

Bazan J F. Structural design and molecular evolution of a cytokine receptor superfamily. Proc Natl Acad Sci USA. 1990 Se; 87(18):6934-8.
Bravo J, Heath J K. Receptor recognition by gp130 cytokines. EMBO J. 2000 Jun. 1; 19(11):2399-411.
Brudno M., Bioinformatics 2003, 19 Suppl 1:154-162
Carlezon W A Jr, Nestler E J, Neve R L. Herpes simplex virus-mediated gene transfer as a tool for neuropsychiatric research. Crit. Rev Neurobiol. 2000; 14(1):47-67.
Carter P J, Samulski R J. Adeno-associated viral vectors as gene delivery vehicles. Int J Mol. Med. 2000 July; 6(1): 17-27.
Chebath J. et al. Interleukin-6 receptor-interleukin-6 fusion proteins with enhanced interleukin-6 type pleiotropic activities. European Cytokine Network, December 1997, vol. 8, no. 4, pp. 359-365
Chang Li, Gay E E. The molecular genetics of lentiviral vectors—current and future perspectives. Curr Gene Ther. 2001 September; 1(3):237-51.
Davis S, Aldrich T H, Valenzuela D M, Wong V V, Furth M E, Squinto S P, Yancopoulos G D. The receptor for ciliary neurotrophic factor. Science. 1991 Jul. 5; 253(5015):59-63.
Deller M C, Hudson K R, Ikemizu S, Bravo J, Jones E Y, Heath J K. Crystal structure and functional dissection of the cytostatic cytokine oncostatin M. Structure Fold Des. 2000 Aug. 15; 8(8):863-74.
Fischer M, Goldschmitt J, Peschel C, Brakenhoff J P, Kallen K J, Wollmer A, Grotzinger J, Rose-John S. I. A bioactive designer cytokine for human hematopoietic progenitor cell expansion. Nat. Biotechnol. 1997 February; 15(2): 142-5.
Heinrich P C, Behrmann I, Haan S, Hermanns H M, Muller-Newen G, Schaper F. Principles of interleukin (IL)-6-type cytokine signalling and its regulation. Biochem J. 2003 Aug. 15; 374(Pt 1):1-20.
Heller M. J. DNA microarray technology: devices, systems, and applications. Annu. Rev. Biomed. Eng. 2002, 4:129-53
Kallen K. J. et al. The therapeutic potential of interleukin-6 hyperagonists and antagonists. Expert Opinion on Investigational Drugs (1997 March), vol. 6, No. 3, pp. 237-266
Kubler H, Vieweg J. Vaccines in renal cell carcinoma. Semin. Oncol. (2006) 33(5):614-624.
Lieschke G J, Rao P K, Gately M K, Mulligan R G. Bioactive murine and human interleukin-12 fusion proteins which retain antitumour activity in vivo. Nat. Biotechnol. 1997 January; 15(1): 35-40.
Lindemann D, Patriquin E, Feng S, Mulligan R C. Versatile retrovirus vector systems for regulated gene expression in vitro and in vivo. Mol Med. 1997 July; 3(7):466-76.
Marz P, Otten U, Rose-John S, Neural activities of IL-6-type cytokines often depend on soluble cytokine receptors. Eur J. Neurosci. 1999 September; 11(9):2995-3004.
McDonald N Q, Panayotatos N, Hendrickson W A. Crystal structure of dimeric human ciliary neurotrophic factor determined by MAD phasing. EMBO J. 1995 Jun. 15; 14(12):2689-99.
Pflanz S, Tacken I, Grotzinger J, Jacques Y, Minvielle S, Dahmen H, Heinrich P C, Muller-Newen G. A fusion protein of interleukin-11 and soluble interleukin-11 receptor acts as a superagonist on cells expressing gp130. FEBS Lett. 1999 Apr. 30; 450(1-2): 117-22.
Robinson R C, Grey L M, Staunton D, Vankelecom H, Vernallis A B, Moreau J F, Stuart D I, Heath J K, Jones E Y. The crystal structure and biological function of leukemia inhibitory factor: implications for receptor binding. Cell. 1994 Jul. 1; 77(7): 1101-16.
Rose-John S, Schooltink H, Lenz D, Hipp E, Dufhues G, Schmitz H, Schiel X, Hirano T, Kishimoto T, Heinrich P C. Studies on the structure and regulation of the human hepatic interleukin-6 receptor. Eur J. Biochem. 1990 May 31; 190(1):79-83.
Russo G., Zegar C. and Giordano A. Advantages and limitations of microarray technology in human cancer. Oncogene. 2003, 22:6497-507
Somers W, Stahl M, Seehra J S. 1.9 A crystal structure of interleukin 6: implications for a novel mode of receptor dimerization and signaling. EMBO J. 1997 Mar. 3; 16(5):989-97.
Springer M L, Chen A S, Kraft P E, Bednarski M, Blau H M. VEGF gene delivery to muscle: potential role for vasculogenesis in adults. Mol Cell 1998 November; 2(5):549-58.
Thompson, J. D., Higgins, D. G., Gibson, T. J. (1994) Nucleic Acids Res. 22, 4673-80
Türeci O., Sahin U. and Pfreundschuh M. Serological analysis of human tumor antigens: molecular definition and implications. Mol Med. Today. 1997, 3:342-349
Wells J A. Binding in the growth hormone receptor complex. Proc Natl Acad Sci USA. 1996 Jan. 9; 93(1):1-6.
Wiznerowicz M, Fong A Z, Mackiewicz A, Hawley R G. Double-copy bicistronic retroviral vector platform for gene therapy and tissue engineering: application to melanoma vaccine development. GeneTher. 1997 October; 4(10):1061-8.

1. A composition comprising:
1) one or more first cells modified to express a first hyper-cytokine, and
2) one or more second cells modified to express a second hyper-cytokine, wherein said second cells are different from said first cells.

2. The composition of claim 1, wherein the first and/or second hyper-cytokine is a fusion protein comprising a cytokine receptor and a cytokine.

3. The composition of claim 2,
wherein the cytokine receptor is independently selected from
(a) the group consisting of sIL-6R, sIL-11R, OSM-R, CNTF-R, and CT-I-R; or
(b) a polypeptide exhibiting at least 90% sequence identity to a polypeptide according to (a); and
wherein the cytokine is independently selected from
(c) the group consisting of IL-6, IL-11, OSM, CNTF, and CT-I; or
(d) a polypeptide exhibiting at least 90% sequence identity to a polypeptide according to (c),
wherein the hyper-cytokine has hyper-cytokine activity.

4. The composition of claim 2, wherein the cytokine receptor and the cytokine are directly linked or linked by a peptide linker.

5. The composition of claim 1, wherein the hyper-cytokine is a fusion protein comprising
(a) an IL-6R part exhibiting at least 90% sequence identity to human soluble IL-6 receptor (sIL-6R), wherein said sequence identity is calculated over the entire length of the polypeptide sequence from P113 to A323 of SEQ ID NO: 1,
(b) an IL-6 part exhibiting at least 90% sequence identity to human interleukin-6 (IL-6), wherein said sequence identity is calculated over the entire length of the polypeptide sequence from P29 to M212 of SEQ ID NO: 2, and
(c) optionally a peptide linker;
having hyper-cytokine activity
or
(a) an IL-11R part exhibiting at least 90% sequence identity to human soluble IL-11 receptor (sIL-11R), wherein said sequence identity is calculated over the entire length of the polypeptide sequence from M1 to Q365 of SEQ ID NO: 3,
(b) an IL-11 part exhibiting at least 90% sequence identity to human interleukin-11 (IL-11), wherein said sequence identity is calculated over the entire length of the polypeptide sequence from A19 to L199 of SEQ ID NO: 4, and
(c) optionally a peptide linker
having hyper-cytokine activity.

6. The composition of claim 1, wherein the hyper-cytokine is
(a) a polypeptide having the amino acid sequence according to SEQ ID NO: 5, SEQ ID NO: 6, SEQ ID NO: 7, SEQ ID NO: 8 or SEQ ID NO: 9; or
(b) a polypeptide exhibiting at least 90% sequence identity to a polypeptide according to (a) and having hyper-cytokine activity.

7. The composition of claim 1, wherein the first cell and/or the second cells are allogenic cells.

8. The composition of claim 1, wherein the second cells have a different HLA type than the first cells.

9. The composition of claim 1, wherein at least one of the first cells and/or the second cells are tumour cells.

10. The composition of claim 9, wherein the tumour cells are s independently elected for the first and second cells from the group consisting of melanoma cells, renal carcinoma cells, prostate cancer cells, colon cancer cells, lung cancer cells, pancreas cancer cells, liver cancer cells, brain cancer cells, head and neck cancer cells, and sarcoma cells.

11. The composition of claim 9, wherein the first tumour cells are Mich1, deposited under accession number DSM ACC2837 with the “Deutsche Sammlung von Mikroorganismen and Zellkulturen” (DSMZ) and the second tumour cells are Mich2, deposited under accession number DSM ACC2838 with DSMZ.

12. The composition of claim 1, wherein the first cells are Mich1-H6, deposited under accession number DSM ACC2839 with DSMZ.

13. The composition of claim 1, wherein the second cells are Mich2-H6, deposited under accession number DSM ACC 2840 with DSMZ.

14. The composition of claim 1, wherein the proliferation of the first and/or the second cells has been inhibited.

15. The composition of claim 14, wherein the proliferation has been inhibited by radioactive radiation or chemical cross-linking.

16. The composition of claim 1 comprising one or more additional cells, which are different from the first and/or the second cells.

17. The composition of claim 16, wherein said one or more additional cells are one or more additional tumour cells.

18. The composition of claim 16, wherein the proliferation of the one or more additional cells has been inhibited.

19. The composition of claim 18, wherein the proliferation of the one or more additional cells has been inhibited by radioactive radiation or chemical cross-linking.

20. The composition according to claim 16, wherein at least one cell of the one or more additional cells has been modified to express a cytokine, a cytokine receptor or a hypercytokine.

21. (canceled)

22. A pharmaceutical composition comprising a composition according to claim 1 additionally comprising pharmaceutically acceptable diluents, carriers, excipients, fillers, binders, lubricants, glidants, disintegrants, adsorbents, and/or preservatives.

23.-25. (canceled)

26. A method for the treatment or prevention of cancer, comprising administering to a subject in need thereof an amount effective of a composition according to claim 1 for the treatment or prevention of cancer.

27. The method of claim 26, wherein the cancer is melanoma or renal cell, carcinoma, prostate cancer, colon cancer, lung cancer, pancreas cancer, liver cancer, brain cancer, head and neck cancer, or sarcoma.

28. The method of claim 26, wherein the method is for the treatment of a patient having a partially or completely different HLA type than the first allogenic cell and/or the second allogenic cell.
